(19) United States
(12) Patent Application Publication
(10) Pub. No.: US 2016/0051278 A1
(43) Pub. Date: Feb. 25, 2016

(54) Surgical Snare Device

(71) Applicant: EndoChoice, Inc., Alpharetta, GA (US)

(72) Inventors: Charlie Jacobs, Loganville, GA (US); Lanita Cox, Suwanee, GA (US); Brian Lynch, Huntington, NY (US); Chee Kong Teo, Alpharetta, GA (US)

(21) Appl. No.: 14/834,232

(22) Filed: Aug. 24, 2015

Related U.S. Application Data
(63) Continuation-in-part of application No. 14/162,691, filed on Jan. 23, 2014.
(60) Provisional application No. 61/755,971, filed on Jan. 23, 2013.

(51) Int. Cl. A61B 17/3205

(52) U.S. Cl. CPC A61B 17/32056; A61B 18/082

(57) ABSTRACT

A surgical snare device for use in polypectomy procedures includes a snare loop capable of being deployed with a plurality of precise internal diameters. The device includes a handle with first and second handle assemblies and a collar. The first handle assembly has multiple numerical markings on a side. The second handle assembly is slidable along the first handle assembly to deploy the snare loop from an elongate tubular sheath attached to the distal end of the first handle assembly. When the second handle assembly is slid to a position such that it aligns with one of the numerical markings on the first handle assembly, the snare loop will be deployed with an internal diameter in millimeters that corresponds precisely to the numerical marking. For example, sliding the second handle assembly to a ‘10’ marking will deploy a snare loop having an internal diameter of 10 mm. The collar is positioned just distal to the second handle assembly and is also slidable along the first handle assembly. The collar functions as a depth stop and prevents further distal movement of the second handle assembly.

### CROSS-REFERENCE

The present application is a continuation-in-part application of U.S. patent application Ser. No. 14/162,691, entitled “Surgical Snare Device” and filed on Jan. 23, 2014, which relies on U.S. Provisional Patent Application No. 61/755,971, of the same title and filed on Jan. 23, 2013, for priority. The aforementioned applications are herein incorporated by reference in their entirety.

### FIELD

The present specification relates generally to surgical instruments. More particularly, the present specification relates to a surgical snare device used to remove polyps wherein the snare is capable of being deployed with a plurality of precise loop diameters.

### BACKGROUND

Surgical snares are used to remove small masses of tissue that project outward or upward from the normal surface level of hollow organs or body cavities. These masses of tissue are referred to as polyps and the procedure to remove them using a surgical snare is termed a polypectomy. Polypectomy procedures are often performed in the colon. A polypectomy snare device typically includes a long, thin sheath with a handle at its proximal end and a flexible wire within the sheath that terminates in a snare loop. While within the sheath, the wire loop is stored in a closed, compressed configuration. The wire loop can be advanced through an opening at the distal end of the sheath by manipulating an actuator on the handle. As the wire loop is advanced beyond the distal end of the sheath, it expands into an open configuration.

During a polypectomy procedure, the sheath of the surgical snare device (with the snare loop refracted within) is inserted into a working channel of an endoscope. The surgeon advances the sheath until its distal end passes beyond the distal end of the endoscope. The surgeon then positions the distal end of the sheath proximate a target polyp. An assistant then manipulates the handle to advance the wire loop out of the sheath. With the loop now in the open, expanded configuration, the surgeon passes the loop over and around the polyp such that the wire loop encircles the base or stalk of the polyp. The assistant then manipulates the handle in an opposite direction to draw the wire loop back into the sheath. At the same time, an electrical current is supplied to the wire to sever and cauterize the polyp. The severed polyp can then be removed using the snare device or with a separate surgical instrument.

Current snare devices include a wire loop having a specific size and shape. For example, the wire loop can have an oval, circular, or hexagonal shape. When fully extended, a particular wire loop can have an internal diameter, or maximum loop width, of, for example, 13, 27, or 30 mm. U.S. Pat. No. 8,162,938, assigned to Boston Scientific Scimed, Inc., describes “A medical device, comprising: a sheath having a proximal end, a distal end, and a lumen defined therein; a shaft slidably disposed in the lumen, the shaft having a distal end; an end effector coupled to the distal end of the shaft with a crimp band; a coupling member adhered to the shaft, disposed over the crimp band, and positioned adjacent the end effector, the coupling member having a distal region that extends distally of the distal end of the sheath when the end effector is deployed, and the coupling member having a proximal region that extends proximally of the distal end of the sheath when the end effector is deployed; and wherein the coupling member comprises a shrink tube.”

While current snare devices are effective in removing polyps, they are not without their drawbacks. The exact size of the internal diameter of the wire loop can only be known when the wire loop is fully extended. For example, if a surgeon is using snare device having a 30 mm wire loop, the surgeon can only be certain of the loop's internal diameter when it is fully extended at 30 mm. Less than maximum extension of the 30 mm loop will result in a loop with an internal diameter less than 30 mm, but the exact diameter will not be known, will not be related to the amount of actuation of the handle applied by the physician, and will not be easy to repeatably match that diameter. Therefore, if a surgeon wishes to use a snare loop with a different diameter, he will have to switch out his current snare device for a separate snare device designed with a wire loop having that diameter. This requires a surgeon to have a stock of multiple snare devices with different sizes on hand during a procedure. In addition, current snares tend to become deformed after multiple extensions out of and retractions into the sheath. Multiple passages into and out of the sheath are necessary during multiple polypectomies in a single procedure and have a “banana effect” on the loop, wherein the loop becomes narrower and elongated, losing its oval or circular shape.

Therefore, what is needed is a surgical snare device having a wire loop that can be repeatedly and precisely extended to multiple different internal diameters wherein the device includes a mechanism to indicate the exact diameter to the surgeon. What is also needed is a surgical snare device having a resilient wire loop that can be repeatedly withdrawn into and extended from the sheath without losing its open configuration shape.

### SUMMARY

The present specification discloses a surgical snare device for use with an endoscope, said snare device comprising: a first handle assembly having a cylindrical body, a proximal end, a distal end, and a channel within said cylindrical body, wherein said channel is configured to extend from a first point proximate said proximal end to a second point proximate said distal end; a second handle assembly having a center body with a proximal end and a distal end, said second handle assembly coupled to said first handle assembly and slidably movable along said cylindrical body of said first handle assembly; a tubular sheath having a distal end and a proximal end, wherein said proximal end of said sheath is attached to said distal end of said cylindrical body of said first handle; a rigid shaft having a lumen and extending through said channel, wherein a proximal end of said rigid shaft is attached to said distal end of said center body of said second handle assembly; a drive wire having a proximal end and a distal end, wherein said proximal end of said drive wire is configured to extend coaxially through said lumen and is attached to said proximal end of said rigid shaft, and wherein said proximal ends are attached, through said channel, to said distal end of said center body of said second handle assembly; and, a shaped snare loop having a distal end, a proximal end and a point of attachment of said shaped snare loop to said distal end of said drive wire, wherein, when the shaped snare loop is fully retracted within said tubular sheath, the distal end of the shaped snare loop is spaced from the distal end of the tubular sheath by a first length and wherein said first length is at least 10 mm.

Optionally, said tubular sheath has an outside diameter ranging from 2.4 to 2.5 mm, an inside diameter ranging from 1.5 to 1.6 mm and a length ranging from 2,250 to 2,350 mm. Still optionally, said tubular sheath has a length of 2,306 mm.

Optionally, said rigid shaft has an outside diameter ranging from 1.0 to 1.2 mm and an inside diameter ranging from 0.73 to 0.93 mm. Still optionally, said rigid shaft has an outside diameter of 1.1 mm and an inside diameter of 0.83 mm.

Optionally, said rigid shaft has a length ranging from 138.45 to 140.95 mm.

The drive wire may be coated with a colorless and dry lubricant.

Optionally, said drive wire has a diameter ranging from 0.65 to 0.85 mm and a length ranging from 2,250 to 2,350 mm.

The distal end of said shaped snare loop may be formed by bending a wire to form two wire legs of substantially equal lengths with respective free ends. Optionally, said proximal end of said shaped snare loop corresponds to a first point where the two wires merge, and said point of attachment corresponds to a second point where free ends of said two wire legs are attached to said distal end of said drive wire. The shaped snare loop may have a teardrop shape and may include a v-shaped notch extending distally from said distal end of said shaped snare loop.

Optionally, said two wires are merged at said proximal end of the shaped snare loop using a plurality of laser spot welds. The plurality of laser spot welds may include two laser spot welds.

Alternatively, said free ends of said two wire legs are attached to said distal end of said drive wire by a crimped bushing at said point of attachment.

Optionally, said first length ranges from 16 to 23 mm.

Optionally, the snare device further comprises a second length measured from said distal end of said snare loop when fully deployed to said distal end of said snare loop when fully retracted within said sheath, wherein said second length is greater than said first length.

Optionally, the snare device further comprises a cautery tip housing having a cautery pin therein, said housing attached to said distal end of said cylindrical body of said first handle assembly. A distal end of the rigid shaft may be in electrical communication with said cautery tip. The cautery tip housing may be rotatable coaxially about said cylindrical body of said first handle assembly.

Optionally, said proximal end of said drive wire is crimped with said proximal end of said rigid shaft.

The present specification also discloses a snare loop attached to a drive wire of a snare device to enable retraction and extension of the snare loop from a tubular sheath of said snare device, said snare loop comprising: a distal end formed by bending a wire to form first and second wire legs with respective free ends; a proximal end corresponding to a first point formed by merging said first and second wire legs, wherein said first point is positioned between said distal end and said free ends; a loop formed between said distal and proximal ends, said loop having a teardrop shape when extended; and a second point formed by attaching said free ends to said drive wire, wherein said second point is at a distance from said first point.

Optionally, said first and second wire legs are of equal lengths.

Optionally, said distance between said first and second points ranges from 70 to 80 mm.

Optionally, said first and second wires legs are merged at said proximal end using a plurality of laser spot welds. The plurality of laser spot welds may include two laser spot welds.

Alternatively, said free ends are attached to said drive wire by a crimped bushing at said second point.

Optionally, said first point is located approximately 80 mm into said sheath from a distal end of said sheath when the snare loop is fully retracted.

Optionally, said second point is located approximately 160 mm into said sheath from a distal end of said sheath when said snare loop is fully retracted.

A v-shaped notch may extend distally from said distal end of said snare loop. Optionally, a length of the snare loop ranges between 128 and 138 mm from said v-shaped notch at said distal end to said first point. Still optionally, a length of the snare loop is 133 mm from said v-shaped notch at said distal end to said first point. Optionally, a length of the snare loop ranges between 126 and 136 mm from said distal end to said first point. Still optionally, a length of the snare loop is 131 mm from said distal end of said snare loop to said first point.

Optionally, said snare loop has a width between 28 and 32 mm when said snare loop is in a fully expanded configuration. Still optionally, said snare loop has an operational width of 30 mm when said snare loop is in a fully expanded configuration.

The wire of said snare loop may comprise a plurality of braided wires. The plurality of braided wires may be equal to seven.

Optionally, said drive wire and said snare loop are annealed at a temperature ranging between 475 and 495° C. Optionally, said annealing is performed for approximately 2 hours. Still optionally, said drive wire and snare loop are water quenched for 10 minutes after annealing.

The aforementioned and other embodiments of the present invention shall be described in greater depth in the drawings and detailed description provided below.

### BRIEF DESCRIPTION OF THE DRAWINGS

These and other features and advantages of the present specification will be further appreciated, as they become better understood by reference to the detailed description when considered in connection with the accompanying drawings:

FIG. 1A is an illustration of one embodiment of a snare device of the present specification with a snare loop withdrawn into a sheath;

FIG. 1B is an illustration of one embodiment of a snare loop attached to a flexible drive wire;

FIG. 1C illustrates relaxed and cinched positions of the snare device and the snare loop in accordance with an embodiment;

FIG. 2 is a side view illustration of the embodiment of the snare device of FIG. 1 with the snare loop withdrawn into the sheath, depicting measurement markings printed on the side of the first handle assembly;

FIG. 3 is a side view illustration of the embodiment of the snare device of FIG. 1 with the snare loop withdrawn into the sheath, depicting the collar positioned just distal to the ‘10’ marking on the first handle assembly;

FIG. 4 is a side view illustration of the embodiment of the snare device of FIG. 1 with the snare loop deployed, depicting the second handle assembly advanced to the ‘10’ marking on the first handle assembly;

FIG. 5 is a side view illustration of the embodiment of the snare device of FIG. 1 with the snare loop withdrawn into the sheath, depicting the collar positioned just distal to the ‘20’ marking on the first handle assembly;

FIG. 6 is a side view illustration of the embodiment of the snare device of FIG. 1 with the snare loop deployed, depicting the second handle assembly advanced to the ‘20’ marking on the first handle assembly;

FIG. 7 is a side view illustration of the embodiment of the snare device of FIG. 1 with the snare loop withdrawn into the sheath, depicting the collar positioned just distal to the ‘30’ marking on the first handle assembly;

FIG. 8 is a side view illustration of the embodiment of the snare device of FIG. 1 with the snare loop deployed, depicting the second handle assembly advanced to the ‘30’ marking on the first handle assembly; and,

FIG. 9 is a flowchart illustrating one embodiment of the steps included during a multiple polypectomy procedure using the surgical snare device of the present specification.

### DETAILED DESCRIPTION

The present specification discloses a surgical snare device having a snare loop that is deployable across a range of internal diameters in a precise and controlled manner. The present invention is directed toward multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

The device includes a handle and an elongated tubular sheath attached to the distal end of the handle. The handle comprises a first handle assembly, a second handle assembly, a cautery tip housing, and a collar. The first handle assembly includes a cylindrical body with a proximal end and a distal end, a finger ring at the proximal end, and a channel extending from a point proximate its proximal end to a point proximate its distal end. The cautery tip housing is attached to and rotatable coaxially about, the distal end of the first handle assembly. The second handle assembly includes a center body portion having a proximal end and a distal end with a finger ring positioned on both sides. The second handles assembly and collar are coupled to, and slidably movable along via the channel of, the first handle assembly. The collar includes a proximal end and a distal end and is positioned distal to the second handle assembly. A rigid drive shaft having a lumen within, a proximal end, and a distal end is positioned within the channel. The shaft extends distally through the channel, into the cautery tip housing, wherein it is in electrical contact with the cautery tip. An elongate tubular sheath is attached to the distal end of the first handle assembly and extends distally from the handle. A flexible drive wire, having a proximal end and a distal end, extends distally within the tubular sheath and has a snare loop attached at its distal end. A proximal portion of the flexible drive wire is positioned coaxially within the lumen of the rigid drive shaft along the entire length of the rigid drive shaft such that the proximal end of the flexible drive wire is flush with the proximal end of the rigid drive shaft. The proximal ends of the flexible drive wire and rigid drive shaft are permanently affixed, through mechanical crimping, and the affixed proximal ends are attached to the distal end of the center portion of the second handle assembly through the channel of the first handle assembly. The snare loop rests within the sheath in a collapsed configuration and is deployable, by sliding the second handle assembly distally along the first handle assembly, to an expanded configuration out of the distal end of the sheath.

The first handle assembly includes a plurality of numerical markings along its length. The markings correspond precisely to the internal diameter or width in millimeters of the deployed snare loop when the distal end of the center body of the second handle assembly aligns with said marking. Therefore, when the distal end of the center body of the second handle assembly is aligned with a ‘10’, the deployed snare loop will have an internal diameter of 10 mm. When the distal end of the center body of the second handle assembly is aligned with a ‘20’, the deployed snare loop will have an internal diameter of 20 mm, and so forth. The collar is included to provide a depth stop for the second handle assembly at the desired marking Therefore, when a user desires to have a snare loop with an internal diameter of 10 mm, the collar is slid along the first handle assembly such that its proximal end aligns with the ‘10’ marking so that the second handle assembly, when slid distally, will come to rest with its distal end against the proximal end of the collar.

In one embodiment, the drive wire and snare loop are comprised of multiple braided wires. The snare loop has a teardrop shape with a v-shaped notch in its distal end. The wires of the device are annealed at a precise temperature for a specific amount of time to produce a stable loop shape. The snare loop shape and manufacturing process ensure a flat loop deployment and retraction, thereby reducing the need for repositioning the endoscope during deployment and optimizing precise resection during retraction. The snare loop design also enhances tactile feel during resection, reducing inadvertent premature shearing and enhancing user comfort and efficiency. The teardrop configuration serves to maximize the snare loop internal diameter while minimizing the snare loop length, thereby providing greater maneuverability in confined anatomy. The teardrop configuration also reduces the handle throw, enhancing user comfort.

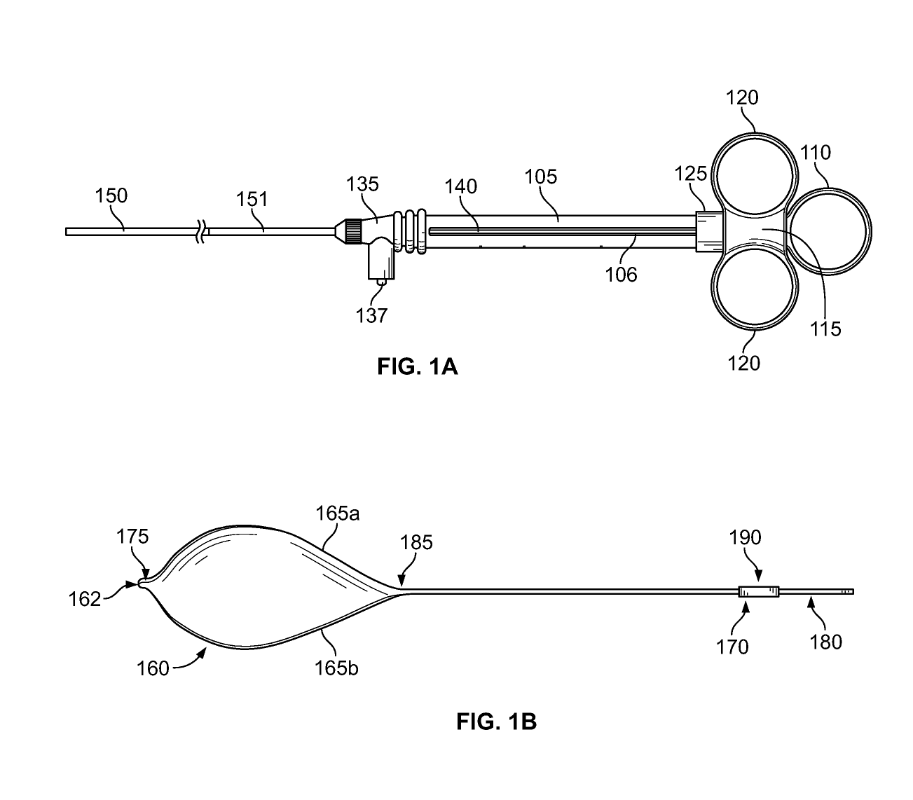

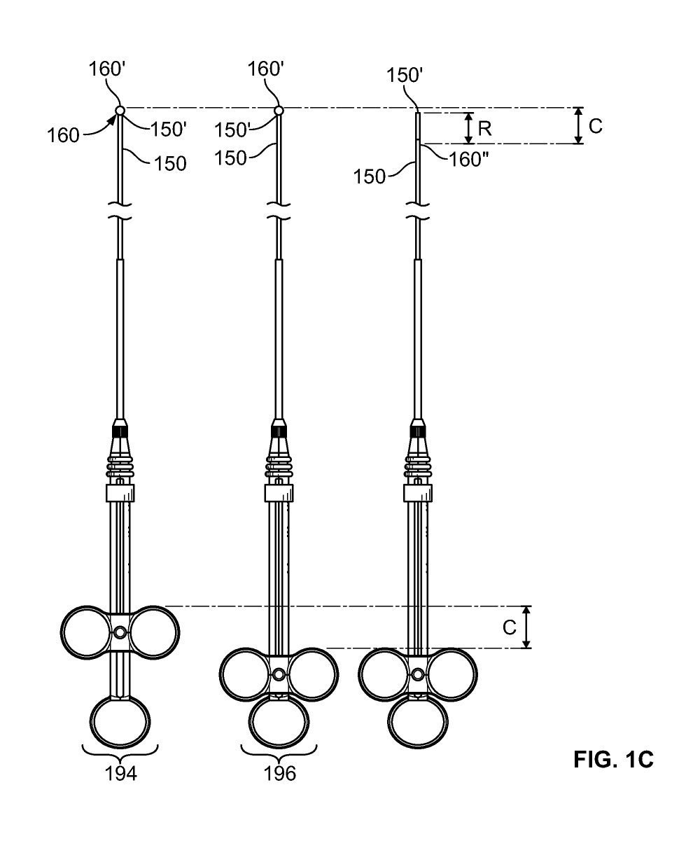

FIG. 1A is an illustration of one embodiment of the snare device of the present specification with a snare loop (not shown) withdrawn into the sheath 150 while FIG. 1B is an illustration of an embodiment of the snare loop 160 attached to a flexible drive wire 180. In the pictured embodiment, the snare device includes a first handle assembly 105 and a second handle assembly 115. The first handle assembly 105 comprises a cylindrical body having a proximal end and a distal end and a channel 106 extending from a point proximate the proximal end to a point proximate the distal end. A finger ring 110 is positioned at the proximal end of the first handle assembly 105. A cautery pin housing 135 with cautery pin 137 is attached to the distal end of the first handle assembly 105. In one embodiment, the cautery pin housing 135 is rotatable coaxially relative to the first handle assembly 105, increasing user comfort and efficiency. The second handle assembly 115 comprises a center portion with a finger ring 120 on each side. The second handle assembly 115 is coupled to the first handle assembly 105 and slidably movable within the channel 106 of the first handle assembly 105. A collar 125, positioned distal to the second handle assembly 115, is also coupled to the first handle assembly 105 and slidably movable within the channel 106 of the first handle assembly 105.

An elongate tubular sheath 150 having a lumen therein is attached to the distal end of the first handle assembly 105 and extends distally through an opening at the distal end of the cautery tip housing 135. The sheath 150 functions as a drive tube for the snare and a drive wire. In various embodiments, the sheath 150 has an outer diameter ranging between 2.4 and 2.5 mm and an inner diameter (of the lumen) ranging between 1.5 and 1.6 mm. In various embodiments, the sheath length is between 2,250 and 2,350 mm. In one embodiment, the sheath length is 2,306 mm. A rigid drive shaft 140 is attached to the distal end of the center portion of the second handle assembly 115 and extends distally through the channel 106 of the first handle assembly 105. A flexible drive wire (such as the flexible drive wire 180 of FIG. 1B) extends distally within the lumen of the sheath 150 from the cautery tip housing 135 to a point proximate the distal end of the sheath 150 where it is attached to a snare loop (such as the snare loop 160 of FIG. 1B). A drive assembly is formed by inserting the proximal end of the flexible drive wire into a lumen of the rigid drive shaft 140 through the distal end of said rigid drive shaft 140. The flexible drive wire is passed coaxially within the lumen and along the entire length of the rigid drive shaft 140 until the proximal end of the flexible drive wire is positioned flush with the proximal end of the rigid drive shaft 140. Thereafter, the drive assembly is mechanically crimped and attached to the distal end of the center portion of the second handle assembly 115 through the channel 106 of the first handle assembly 105.

In one embodiment, the flexible drive wire is comprised of multiple braided wires. In one embodiment, the drive wire includes seven braided wires. In various embodiments, the flexible drive wire is coated with a colorless and dry lubricant to reduce friction between the drive wire, the sheath 150 and the first handle assembly 105. In various embodiments, the lubricant includes any one of polytetrafluoroethylene (PTFE), powder, McLube®, and silicon oil. In an exemplary testing situation, Twizzler® candy was used as a tissue sample. The sample size taken was of 30 mm for each group, one group with lubricated drive wire, and the other without. 100% of the lubricant coated group samples completely sheared through the cutting specimen (that is, the Twizzler candy representing tissue) in less than one minute, while the uncoated group (that is, the group without lubricant coated drive wire) did not shear the Twizzler candy completely even after 2 minutes in a cinched position (shown as position 196 in FIG. 1C, as an example) of the snare loop (such as the snare loop 160 of FIG. 1B).

In various embodiments, the flexible drive wire has a diameter ranging between 0.65 and 0.85 mm and a length ranging between 2,250 and 2,350 mm. In one embodiment, the flexible drive wire has a diameter of 0.75 mm and a length of 2,294 mm. In various embodiments, the rigid drive shaft 140 has an outside diameter ranging between 1.0 and 1.2 mm and an inside diameter ranging between 0.73 and 0.93 mm. In one embodiment, the rigid drive shaft 140 has an outside diameter of 1.1 mm and an inside diameter of 0.83 mm. In various embodiments, the length of the rigid drive shaft 140 is 139.7 mm+/−1.25 mm. In one embodiment, the braided wires of the flexible drive wire and the rigid drive shaft 140 are composed of stainless steel (SS304), Nickel-Titanium (NiTi) or a combination thereof.

The rigid drive shaft 140 continues through the cautery tip housing 135, wherein it is in electrical communication with the cautery tip 137. In one embodiment, a length of the sheath 150 just distal to the cautery tip housing 135 includes a shaft strain relief 151. The shaft strain relief 151 overlays the sheath 150 and provides strain relief to the rigid drive shaft 140 as the drive shaft 140 is advanced into the sheath 150 to deploy the snare loop. The shaft strain relief 151 ensures smooth movement of the drive shaft 140 to facilitate proper deployment of the snare loop. In various embodiments, the shaft strain relief 151 has a length between 90 and 100 mm, an outside diameter between 3.1 and 3.3 mm, and an inside diameter between 2.2 and 2.4 mm. In one embodiment, the shaft strain relief 151 has a length of 100 mm, an outside diameter of 3.2 mm, and an inside diameter of 2.3 mm.

In various embodiments, the snare loop also comprises multiple braided wires. In one embodiment, the snare loop comprises seven braided wires. In various embodiments, the snare loop has a diameter between 0.35 and 0.45 mm. In one embodiment, the snare loop has a diameter of 0.40 mm. Referring now to FIG. 1B, a bent point is mechanically formed to establish the distal end 162 of the loop 160. In one embodiment, the bent point is exactly mid-point of the snare loop wire, thus, forming two legs 165a, 165b of equal lengths extending from the distal end 162 of the loop 160 to the free ends of the loop wire at an attachment/crimping position 170. In one embodiment, the bent point is mechanically stamped into the shape of a teardrop. Thus, in one embodiment, the snare loop 160 has a teardrop shape and includes a v-shaped notch 175 extending outwardly from its distal end 162. In another embodiment, the snare loop 160 has a teardrop shape and does not include the v-shaped notch 175.

Referring to FIGS. 1A and 1B, in one embodiment, the snare loop geometry is defined by the v-shaped notch 175 distally and a merging point 185 proximally. In one embodiment, the location of the merging point 185 is positioned between 70 to 80 mm distal from the attachment/crimping position 170, of both wire legs 165a, 165b. The merging point 185 is defined as the point along the loop wire where the two lengths of the wire are merged to form the proximal end of the loop. This is in contrast to the attachment/crimping position 170 in FIG. 1B, which is defined as the point at the free ends of the loop wire where the two free ends are attached to the flexible drive wire 180. In the prior art, the merging point 185 includes a bushing which is typically crimped onto the wires 165a, 165b. In one embodiment of the present specification, the merging point 185 is laser spot/resistance welded to the wires 165a, 165b, resulting in no raised edge to drag against the sheath 150 as the snare loop 160 deploys. This ensures smooth drive wire movement to facilitate snare loop deployment and retraction in tortuous anatomy. In accordance with an embodiment, two laser spot welds are used at the merging point 185 to ensure a flat loop deployment and retraction, thereby reducing the need for repositioning the endoscope during deployment and optimizing precise resection during retraction. In one embodiment, the merging point 185 is located approximately 80 mm into the sheath 150 from a distal end of the sheath 150 when the snare loop 160 is fully retracted.

In accordance with an embodiment, both legs 165a, 165b of wires at the attachment/crimping position 170 of the snare loop 160 are attached to the flexible drive wire 180 by a crimped bushing 190. In an embodiment, the crimped bushing 190 is located approximately 160 mm into the sheath 150 from the distal end of the sheath 150 when the snare loop 160 is fully refracted, which is further in than what is encountered with prior art snare devices.

During manufacture, in accordance with an aspect, the flexible drive wire and snare loop 160 are annealed at 485° C.+/−10° C. for about two hours concurrent with loop shape formation, and water quenched for 10 minutes. The teardrop shape with notch 175 and annealing process help to maintain the loop configuration during multiple polypectomies in a single procedure, eliminating the ‘banana’ effect.

In various embodiments, the length of the snare loop 160, including the v-shaped notch 175, is between 128 and 138 mm from the attachment/crimping position 170 to its distal end 162. In one embodiment, the length of the snare loop 160, including the v-shaped notch 175, is 133 mm from the attachment/crimping position 170 to its distal end 162. In various embodiments, the length of the snare loop 160 without the v-shaped notch 175 is between 126 and 136 mm from the attachment/crimping position 170 to its distal end 162. In one embodiment, the length of the snare loop 160 without the v-shaped notch 175 is 131 mm from the attachment/crimping position 170 to its distal end 162. In various embodiments, a fully deployed/expanded snare loop 160 has a maximum operational width between 28 and 32 mm. In one embodiment, a fully deployed/expanded snare loop 160 has a maximum operational width of 30 mm.

In various embodiments, the snare device has a working length between 2,250 and 2,350 mm. In one embodiment, the snare device has a working length of 2,300 mm. In various embodiments, the snare device has a handle deployment throw between 110 and 130 mm and a snare deployment throw of 105 to 109 mm. In one embodiment, the snare device has a handle deployment throw of 120 mm and a snare deployment throw of 107 mm.

FIG. 1C illustrates relaxed and cinched states or positions 194, 196 respectively, of the snare device in accordance with various embodiments. The figure shows a refraction length ‘R’ defined as a distance between a distal end 150′ or top of the sheath 150 to a distal end 160″ or top of the snare loop 160 when the snare loop 160 is fully retracted. Also illustrated is a cinched length ‘C’ defined as a distance from the distal end 160′ or top of the snare loop 160 when in fully deployed yet relaxed or no-load position 194 to the distal end 160″ or top of the snare loop 160 when the snare loop 160 is in fully retracted position 196. Thus, the retraction length ‘R’ is part of the cinched length ‘C’. When the snare loop 160 cinches down on a cutting specimen or anomaly, such as a polyp for example, this condition, action or state is modeled, in an embodiment, as a spring force system adhering to Hooke's law, with the following force:

Force=K*C

where K, the system stiffness, is attributed to the following:
Specimen shear properties
Sheath or catheter column stiffness
Drive wire and snare loop stiffness
The shearing properties of the loop wire

In accordance with an embodiment, since the snare device is constructed with an amount of retraction length ‘R’ when the snare is fully retracted, this contributes to a larger cinched length ‘C’ component, which increases force. The force is used to shear the tissue, specimen, or anomaly, and to overcome friction between the sheath and the drive wire. It should be appreciated that if the snare device does not comprise the retraction length ‘R’, the cinched length component ‘C’ would be much smaller as a result of which the force will also be smaller. In some embodiments, the refraction length ‘R’ measures at least 10 mm. In one embodiment, the retraction length ‘R’ measures 20 mm nominal, with a tolerance of +3 mm, −4 mm. Thus, when friction between the sheath and the drive wire is reduced, more of the force is used for shearing the tissue, specimen or anomaly, and less to overcome the static and dynamic coefficient friction of the sheath and drive wire.

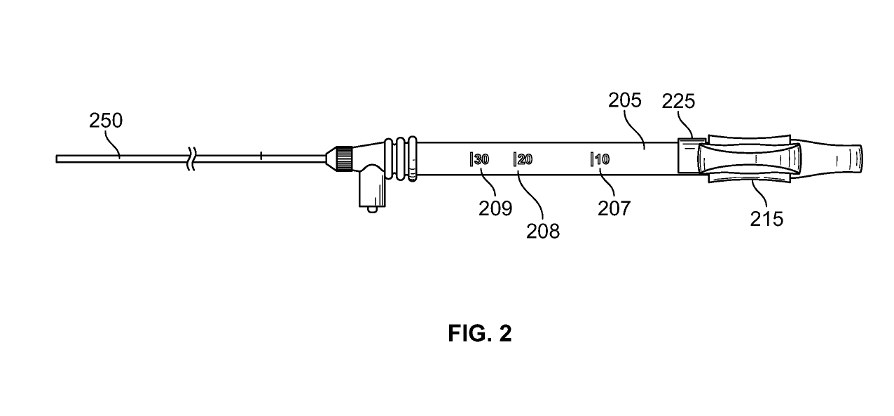

FIG. 2 is a side view illustration of the embodiment of the snare device of FIG. 1 with the snare loop withdrawn into the sheath 250, depicting measurement markings 207, 208, 209 printed on the side of the first handle assembly 205. To deploy the snare loop, the collar 225 and second handle assembly 215 are advanced distally along the first handle assembly 205, thereby advancing the shaft and drive wire and deploying the loop past the distal end of the sheath 250. Each measurement marking 207, 208, 209 on the first handle assembly 205 corresponds precisely to the internal diameter or width of the deployed snare loop in millimeters when the distal end of the second handle assembly 215 has been advanced to one of said markings 207, 208, 209. For example, when the distal end of the second handle assembly 215 is moved distally to the ‘10’ marking 207, the deployed snare loop will have a width, at the widest point in the loop, of 10 mm. When the distal end of the second handle assembly 215 is moved further distally to the ‘20’ marking 208, the deployed snare loop will have a width, at the widest point in the loop, of 20 mm and, when the distal end of the second handle assembly 215 is moved even further distally to the ‘30’ marking 209, the deployed snare loop will have a width, at the widest point in the loop, of 30 mm.

The collar 225 is moved just distal to the desired marking 207, 208, 209 before sliding the second handle assembly 215 and functions as a depth stop for the second handle assembly 215 to indicate to the user that he or she has deployed the snare loop the desired amount. Both the collar 225 and the second handle assembly 215 slide along the first handle assembly 205 with some resistance, thereby preventing unintended movement of either component. In one embodiment, each depth marking is denoted by a visible line or, alternatively, an indentation or a raised marking which, when met by the collar 225, generates a clicking sound to indicate to the physician that the precise denoted depth has been reached. Correlating the depth stops precisely with multiple snare loop internal diameters provides the surgeon with a snare device having the functionality of 2 or more prior art devices, thereby alleviating the need of keeping an inventory of different sized snares. In addition, having the ability to deploy a snare loop with multiple, precise widths provides accurate sizing capability of the target tissue to assist the surgeon with immediate follow-up protocol. The collar functioning as a depth stop also provides hybrid throw characteristics of a traditional snare handle and those of a traditional short throw, providing greater versatility and cost efficiency.

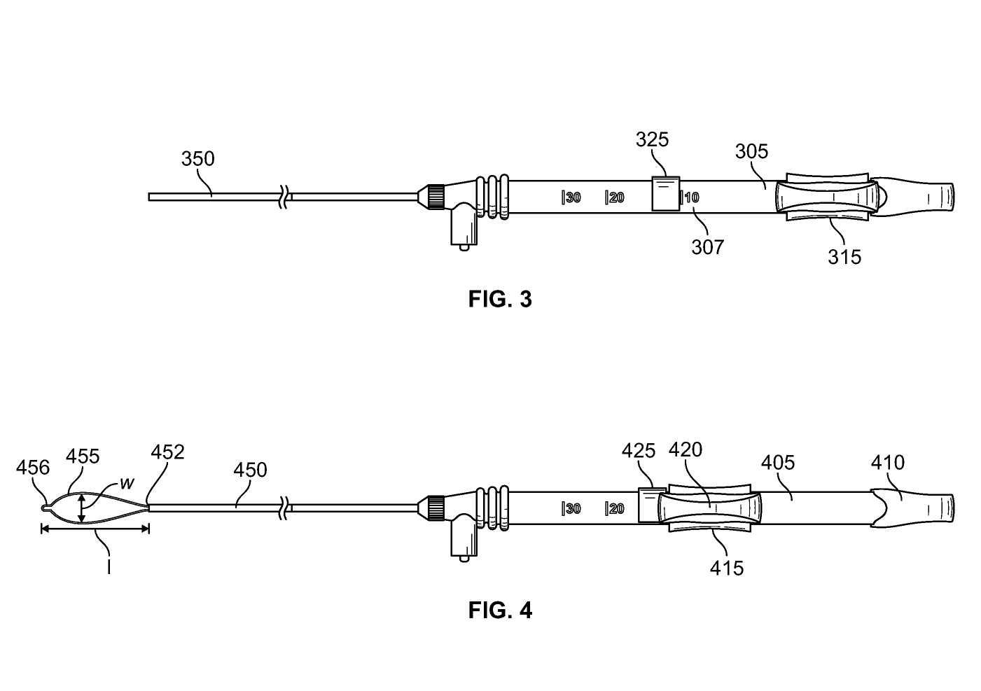

FIG. 3 is a side view illustration of the embodiment of the snare device of FIG. 1 with the snare loop withdrawn into the sheath 350, depicting the collar 325 positioned just distal to the ‘10’ marking 307 on the first handle assembly 305. The second handle assembly 315 is still in its most proximal position and the snare loop is still withdrawn inside the sheath 350. Referring to FIG. 4, the second handle assembly 415 has been slid distally along the first handle assembly 405 until its distal end comes to rest against the proximal end of the collar 425. The collar 425 is in the same position as shown in FIG. 3, though the ‘10’ marking is covered by the second handle assembly 415 in FIG. 4. Advancing the second handle assembly 415 to the position depicted in FIG. 4 deploys the snare loop 455 from the sheath 450 such that the snare loop 455 has an internal diameter or width w of precisely 10 mm, corresponding to the marking on the first handle assembly 405. In addition, when the second handle assembly is at the position depicted in FIG. 4, the snare loop 455 has a length, not including the v-shaped notch 456 discussed below, of 25 mm from the opening 452 at the distal end of the sheath 450 to the distal end of the snare loop 455.

Also visible in FIG. 4 is the v-shaped notch 456 at the distal end of the snare loop. The notch 456 helps the snare loop 455 keep its teardrop shape as it is extended from and retracted into the sheath 450. To deploy the snare loop 455, a user moves the second handle assembly 415 by holding the first handle assembly in place with finger ring 410 and simultaneously using finger rings 420 to slide the second handle assembly 415 distally along the first handle assembly 405. When the snare loop 455 is in the withdrawn position within the sheath 450, the wires of the two ends of the snare loop are positioned parallel to one another. As the snare loop 455 is deployed, the wires twist over each other and then eventually untwist once the snare loop 455 has been deployed to the desired width.

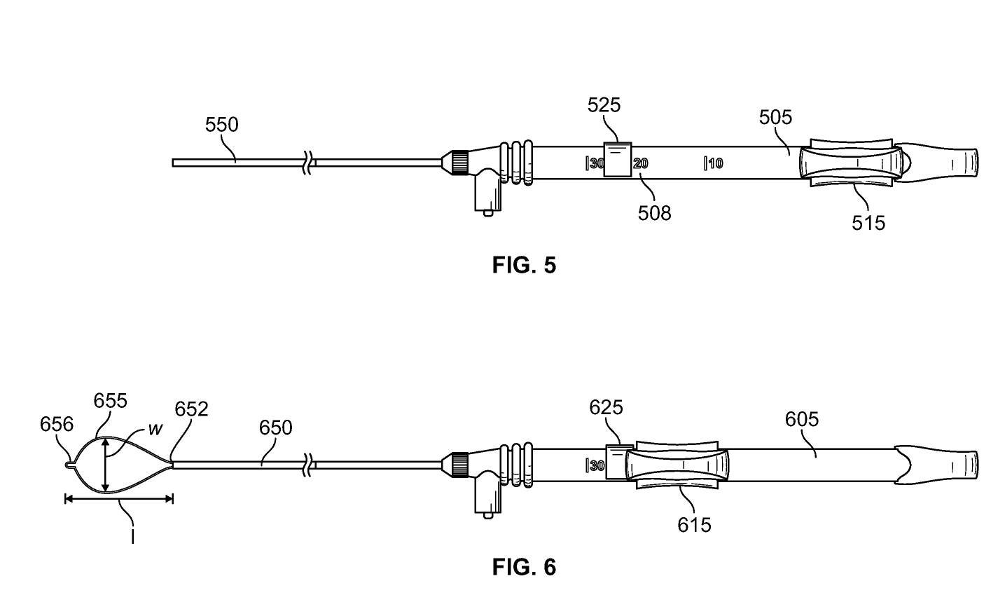

FIG. 5 is a side view illustration of the embodiment of the snare device of FIG. 1 with the snare loop withdrawn into the sheath 550, depicting the collar 525 positioned just distal to the ‘20’ marking 508 on the first handle assembly 505. The second handle assembly 515 is still in its most proximal position and the snare loop is still withdrawn inside the sheath 550. Referring to FIG. 6, the second handle assembly 615 has been slid distally along the first handle assembly 605 until its distal end comes to rest against the proximal end of the collar 625. The collar 625 is in the same position as shown in FIG. 5, though the ‘20’ marking is covered by the second handle assembly 615 in FIG. 6. Advancing the second handle assembly 615 to the position depicted in FIG. 6 deploys the snare loop 655 from the sheath 650 such that the snare loop 655 has an internal diameter or width w of precisely 20 mm, corresponding to the marking on the first handle assembly 605. In addition, when the second handle assembly is at the position depicted in FIG. 6, the snare loop 655 has a length, not including the v-shaped notch 656, of 42 mm from the opening 652 at the distal end of the sheath 650 to the distal end of the snare loop 655.

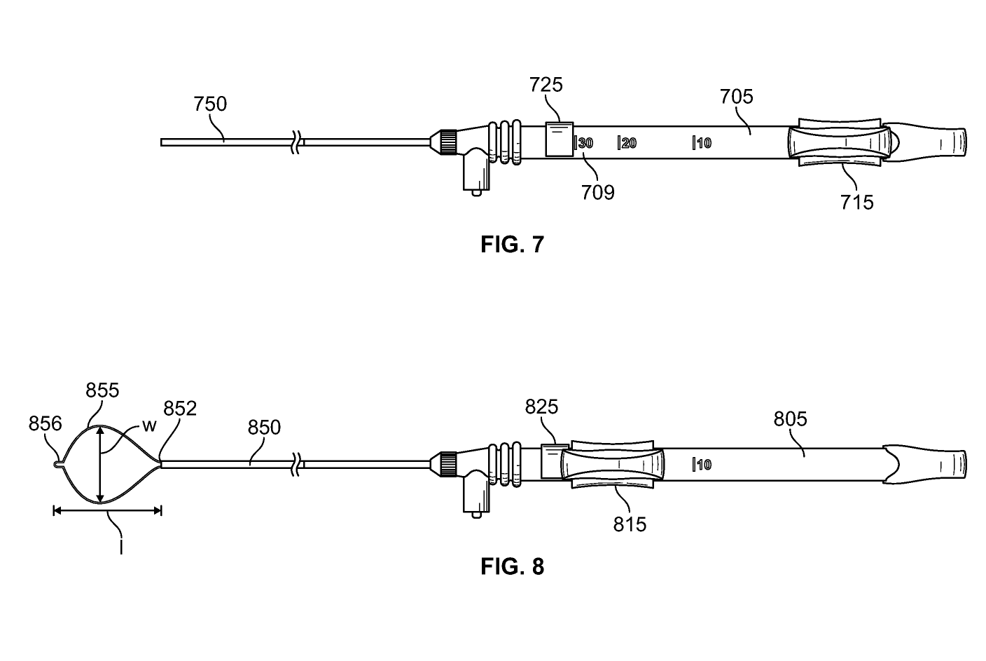

FIG. 7 is a side view illustration of the embodiment of the snare device of FIG. 1 with the snare loop withdrawn into the sheath 750, depicting the collar 725 positioned just distal to the ‘30’ marking 709 on the first handle assembly 705. The second handle assembly 715 is still in its most proximal position and the snare loop is still withdrawn inside the sheath 750. Referring to FIG. 8, the second handle assembly 815 has been slid distally along the first handle assembly 805 until its distal end comes to rest against the proximal end of the collar 825. The collar 825 is in the same position as shown in FIG. 7, though the ‘30’ marking is covered by the second handle assembly 815 in FIG. 8. Advancing the second handle assembly 815 to the position depicted in FIG. 8 deploys the snare loop 855 from the sheath 850 such that the snare loop 855 has an internal diameter or width w of precisely 30 mm, corresponding to the marking on the first handle assembly 805. In addition, when the second handle assembly is at the position depicted in FIG. 8, the snare loop 855 has a length, not including the v-shaped notch 856, of 51 mm from the opening 852 at the distal end of the sheath 850 to the distal end of the snare loop 855.

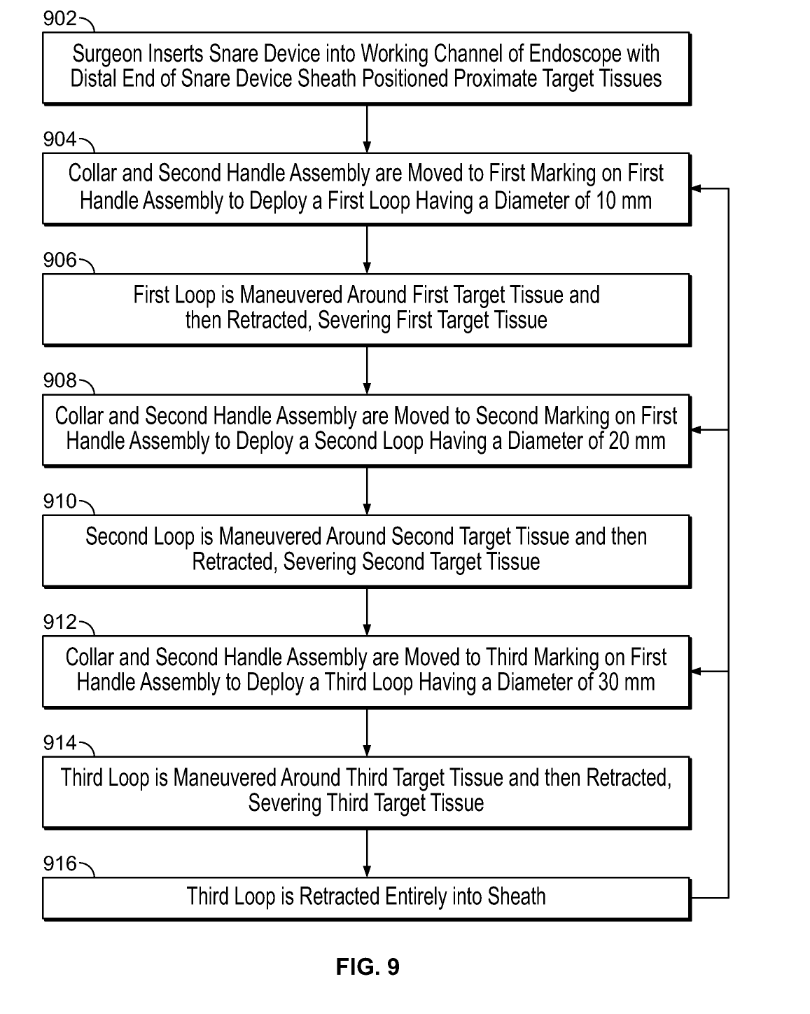

FIG. 9 is a flowchart illustrating one embodiment of the steps included during a multiple polypectomy procedure using the surgical snare device of the present specification. At step 902, a surgeon inserts the snare device into a working channel of an endoscope with the distal end of the sheath positioned proximate target tissues. Then, at step 904, the collar and second handle assembly are moved to the first marking on the first handle assembly to deploy a first loop having a diameter of 10 mm. The first loop is maneuvered around a first target tissue and then retracted, severing the first target tissue at step 906. At step 908, the collar and second handle assembly are moved to the second marking on the first handle assembly to deploy a second loop having a diameter of 20 mm. The second loop is maneuvered around a second target tissue and then refracted, severing the second target tissue at step 910. At step 912, the collar and second handle assembly are moved to the third marking on the first handle assembly to deploy a third loop having a diameter of 30 mm. The third loop is maneuvered around a third target tissue and then retracted, severing the third target tissue at step 914. At step 916, the third loop is retracted entirely into the sheath. At this point, the surgeon can redeploy the snare with a diameter of 10 mm at step 904, with a diameter of 20 mm at step 908, or with a diameter of 30 mm, at step 912 and continue on. The deployment and retraction can be done repeatedly and the loop dimensions will be same each time in correspondence with the marking on the first handle assembly.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A snare loop attached to a drive wire of a snare device to enable retraction and extension of the snare loop from a tubular sheath of said snare device, said snare loop comprising:
a distal end formed by bending a wire to form first and second wire legs with respective free ends;
a proximal end corresponding to a first point formed by merging said first and second wire legs, wherein said first point is positioned between said distal end and said free ends;
a loop formed between said distal and proximal ends, said loop having a teardrop shape when extended; and
a second point formed by attaching said free ends to said drive wire, wherein said second point is at a distance from said first point.

2. The snare loop of claim 1, wherein said first and second wire legs are of equal lengths.

3. The snare loop of claim 1, wherein said distance between said first and second points ranges from 70 to 80 mm.

4. The snare loop of claim 1, wherein said first and second wires legs are merged at said proximal end using a plurality of laser spot welds.

5. The snare loop of claim 4, wherein said plurality of laser spot welds include two laser spot welds.

6. The snare loop of claim 1, wherein said free ends are attached to said drive wire by a crimped bushing at said second point.

7. The snare loop of claim 1, wherein said first point is located approximately 80 mm into said sheath from a distal end of said sheath when the snare loop is fully retracted.

8. The snare loop of claim 1, wherein said second point is located approximately 160 mm into said sheath from a distal end of said sheath when said snare loop is fully retracted.

9. The snare loop of claim 1, wherein a v-shaped notch extends distally from said distal end.

10. The snare loop of claim 9, wherein a length of the snare loop ranges between 128 and 138 mm from said v-shaped notch at said distal end to said first point.

11. The snare loop of claim 9, wherein a length of the snare loop is 133 mm from said v-shaped notch at said distal end to said first point.

12. The snare loop of claim 1, wherein a length of the snare loop ranges between 126 and 136 mm from said distal end to said first point.

13. The snare loop of claim 1, wherein a length of the snare loop is 131 mm from said distal end of said snare loop to said first point.

14. The snare loop of claim 1, wherein said snare loop has a width between 28 and 32 mm when said snare loop is in a fully expanded configuration.

15. The snare loop of claim 1, wherein said snare loop has an operational width of 30 mm when said snare loop is in a fully expanded configuration.

16. The snare loop of claim 1, wherein said wire of said snare loop comprises a plurality of braided wires.

17. The snare loop of claim 16, wherein said plurality of braided wires is equal to seven.

18. The snare loop of claim 1, wherein said drive wire and said snare loop are annealed at a temperature ranging between 475 and 495° C.

19. The snare loop of claim 18, wherein said annealing is performed for approximately 2 hours.

20. The snare loop of claim 19, wherein said drive wire and snare loop are water quenched for 10 minutes after annealing.
